(19) United States
(12) Patent Application Publication
(10) Pub. No.: US 2005/0234481 A1
(43) Pub. Date: Oct. 20, 2005

(54) Suture cutting device

(75) Inventor: David F. Waller, Winston-Salem, NC (US)

Correspondence Address: BRINKS HOFER GILSON & LIONE, P.O. BOX 10395, CHICAGO, IL, 60610, US

(73) Assignee: Wilson-Cook Medical Inc.

(21) Appl. No.: 11/072,705

(22) Filed: Mar. 4, 2005

Related U.S. Application Data
(60) Provisional application No. 60/558,234, filed on Mar. 31, 2004.

(51) Int. Cl. A61B 17/04

(52) U.S. Cl. 606/148

(57) ABSTRACT

A surgical device for manipulating and cutting a suture, including methods for making the device and methods for using the device in minimally invasive and general surgical procedures.

### CROSS-REFERENCE TO RELATED APPLICATIONS

This application claims priority to U.S. Provisional Application Ser. No. 60/558,234, filed Mar. 31, 2004.

### TECHNICAL FIELD

The present invention relates generally to surgical instruments. More particularly, the invention concerns a device for cutting sutures and is well-configured for use in minimally invasive surgical techniques.

### BACKGROUND

The invention has particular use with respect to cutting sutures in a gastrointestinal tract or other body lumen.

Cutting of sutures is commonly required in a variety of situations during or after a surgical procedure. In a general surgery setting, this is normally accomplished using a pair of surgical scissors. However, in minimally invasive surgical techniques such as endoscopy, laparoscopy, arthroscopy and the like, use of traditional surgical scissors is impractical or impossible. This is because the scissors cannot be inserted and manipulated through a surgical access port (such as a body orifice or an incision). Likewise, the scissors cannot be inserted and manipulated through a minimally invasive surgical tool.

In minimally invasive surgical procedures, only a small incision is made in the patient for introduction and use of surgical tools. Alternatively, the surgical work is conducted through an existing body orifice. Consequently, the surgeon's access to the actual operating site inside the patient is restricted. Therefore, specialized instruments are needed for working efficiently and effectively in a minimally invasive surgical environment.

Various types of endoscopy-related and other minimally invasive surgical instruments are known in the art. One type of instrument generally comprises a slender tube containing a push rod that is axially movable within the tube by means of a handle or trigger-like actuating means. An end effector is provided at the distal end of the tube and is coupled to the push rod such that axial movement or rotational movement of the push rod is translated to, respectively, axial or pivotal movement of the end effector. End effectors may take the form of scissors, grippers, cutting jaws, forceps, and the like. Because of their very small size and the requirements of strength and/or sharpness, end effectors are difficult to manufacture and are typically formed of forged stainless steel, or are cast from plastic, bronze, or from another alloy or superalloy. In addition, the requirement for sterility, precision manufacturing, and particularized applications often necessitates complex designs using specialized materials. As a result, end effector tools are commonly quite expensive to manufacture. Thus, there is a need for basic but effective surgical tools that are adaptable to the specialized needs of endoscopy and other minimally invasive surgery and that are relatively inexpensive to manufacture. Surgical tools that are reusable serve to lower the cost of treatment. Reusable surgical tools need to be constructed with specialized designs and materials suitable for multiple sterilizations.

Currently there are several devices and techniques for cutting sutures while a physician is suturing tissue within a body cavity during endoscopic surgical procedures, or removing sutures already in place. For example, the physician will use endoscopic scissors or sharp biopsy forceps to cut sutures for removal. Using either of these devices poses a risk of trauma to tissue underlying the sutures. The tip or cutting means of scissors can puncture, tear, or cut underlying tissues. Biopsy forceps can cause the same damage, or—if the opposed cutting edges of the forceps do not immediately sever the suture—the tension placed on the suture to be cut can tear or otherwise damage the tissue holding the suture as the suture is pulled against it. Moreover, this can cause damage to tissue which is already under stress due to placement of the suture or the associated surgical procedure. Use of forceps and scissors also poses an increased risk of damage to the endoscope and, because of multiple moving parts, an increased risk of malfunction or breakage requiring retrieval of pieces from inside the patient. In addition, and in part because of the aforementioned risks, these tools require a high level of skill and dexterity, complicating the already delicate task of endoscopic surgery.

What is needed is an minimally invasive surgical device that is relatively easy to manipulate and that can safely cut sutures without damaging the underlying tissue. A device that can be used both for endoscopic and general/traditional surgical removal of sutures would provide an even greater advantage to the field.

### BRIEF SUMMARY

The foregoing problems are solved and a technical advance is achieved in the present invention which provides a device for safely manipulating—including cutting—sutures through an endoscope or in another minimally invasive or a general surgical setting without damaging underlying tissue. The embodiments described herein are useful in general surgical applications as well as in the specialized applications of endoscopic and other minimally invasive surgery. The invention will be relatively inexpensive to manufacture, providing an advantage for physicians, patients, and insurers. Moreover, the embodiments described herein are not as complicated to manufacture or use as is prior art technology, providing extra advantages in cost and in ease of integration to general and minimally invasive surgical practices. Additionally, some embodiments are adaptable to multiple uses following sterilization, conferring a further cost advantage without sacrificing safety.

A first aspect of the invention is a device for manipulating a suture, including cutting the suture. In a first embodiment, the device has an elongate member having a proximal end, a distal end, a main body extending between the proximal and distal ends, and a laterally indented surface near the distal end. The device also has a cutting structure disposed in the indented surface. In a further embodiment of the device, the elongate member has structure in place for attaching the proximal end to a structure for inserting the elongate member into a body lumen (e.g., a catheter, cannula, endoscope working channel). In another further embodiment, the cutting structure is mounted such that at least one portion of the cutting structure protrudes beyond the indented surface, but does not protrude beyond an outermost periphery of the elongate member. In yet another further embodiment, the device also includes a component for opposing the cutting structure and a mechanism configured for advancing the component for opposing the cutting structure toward the cutting edge. The component for opposing the cutting structure has a leading surface, and is slidable along an internal or external surface of the elongate member. The leading surface of the component for opposing the cutting structure is resistant to deforming force. In still another further embodiment, the indented surface includes a curvilinear surface. In still yet another further embodiment, the indented surface includes an angular surface. In a particular embodiment, the cutting structure is a surgical grade steel blade.

A second aspect of the invention is a method for making the device described above. In a first embodiment, the method includes forming an elongate member having a proximal end, a distal end, and a main body extending between the proximal and distal ends. The method also includes forming an indented surface near the distal end and disposing a cutting structure in the indented surface. In one embodiment, forming the elongate member includes machining the elongate member from metal. In another embodiment, forming the elongate member includes molding the elongate member from plastic. Yet another embodiment includes shaping an indented surface in the elongate member. In still another embodiment, forming the elongate member includes molding the elongate member in a cast shaped to form an indented surface. In still yet another embodiment, a step of disposing a cutting structure in the indented surface is included, wherein the elongate member is formed around the cutting structure such that the cutting structure is disposed in the indented surface. In another embodiment, the step of disposing a cutting structure in the indented surface includes forming the cutting structure from a material of the elongate member such that the cutting structure is disposed in an indented surface. Yet another embodiment of disposing a cutting structure in the indented surface includes using a connecting mechanism to affix the cutting structure in the indented surface. In one set of embodiments, the cutting structure is a surgical grade steel blade.

A third aspect of the invention is a method for using a device as described above for cutting of sutures in a body lumen in a general surgical setting. This method includes a step of positioning the device near a suture. In particular, the method includes positioning the distal end of the elongate member such that the suture traverses the indented surface adjacent the cutting edge. Further, the method includes advancing the component for opposing the cutting structure toward the cutting structure such that the leading surface of the component directs the suture into contact against the cutting structure, and then further advancing the component for opposing the cutting structure so that force from the leading-surface-directed contact of the suture against the cutting structure severs the suture.

A fourth aspect of the invention is a method for using a device as described above to cut sutures in a minimally invasive surgical setting. This method includes introducing the device into a body lumen in conjunction with a minimally invasive surgical device and positioning the device near a suture. In particular, the method includes positioning the distal end of the elongate member such that the suture traverses the indented surface adjacent the cutting edge. Further, the method includes advancing the component for opposing the cutting structure toward the cutting structure such that the leading surface directs the suture into contact against the cutting structure, and then further advancing the component for opposing the cutting structure so that force from the leading-surface-directed contact of the suture against the cutting structure severs the suture. It is contemplated that the minimally invasive surgical device will be selected from a group including anuscope, arthroscope, bronchoscope, choledoscope, colonoscope, cystoscope, duodenoscope, earscope, endoscope, endotrachealscope, esophagoscope, hysteroscope, laparoscope, laryngoscope, nasosinuscope, nephroscope, otoscope, pancreatoscope, pelviscope, proctorscope, rectoscope, resectoscope, rhinoscope, sigmoidoscope, sinuscope, thoracoscope, ureteroscope, or another such device.

One specific example of an embodiment is a device for manipulating a suture that includes a substantially cylindrical elongate shaft. The shaft has a proximal end, a rounded distal end, and a main body extending between the proximal and distal ends. The shaft also has an indented surface located near the distal end and having an arched shape wherein is disposed a cutting structure having a cutting edge. The cutting structure is affixed in a distal portion of the arched indentation such that the cutting edge does not protrude beyond an outermost periphery of the elongate member, and such that the cutting edge is oriented toward the proximal end. The shaft is sized and shaped for introduction into a body lumen in conjunction with a minimally invasive surgical device. The device also includes an introducing structure (e.g., a catheter shaft) for inserting the shaft into a body lumen, wherein the introducing structure is attached to the proximal end of the shaft. Alternatively, the introducing structure is integrally part of the elongate shaft of the device. In addition, the device has an oversleeve comprising a leading surface shaped and sized so as to be frictionally slidable over the outer surface of the device shaft.

Use of the present device presents several advantages. Since it is usable with an endoscope, laparoscope, arthroscope or similar device, access to sutures inside of a patient may be accomplished by minimally invasive means. During manipulation of sutures through an endoscope, the device provides for a method of cutting a suture with minimized risks to the underlying tissue in a patient and to the endoscope posed by other devices used in manipulating and cutting sutures, such as endoscopic scissors. In addition, the device is readily adaptable to general surgical applications where its precise, controlled method of function confers an advantage over existing technology and techniques. The described embodiments provide a solution to the problem of how to safely manipulate and cut sutures in a body lumen of a patient. The advantages of the present invention are best understood in view of the following drawings and description of embodiments.

### BRIEF DESCRIPTION OF THE DRAWINGS

FIG. 1 is a side view of one embodiment of a suture cutting device;

FIGS. 2a-e are views of one embodiment of a method of using a suture cutting device;

FIG. 2a is a side view of the suture cutting device with a suture in place;

FIG. 2b is a top view of the device embodiment with a suture in place for cutting;

FIG. 2c is a perspective view of the device embodiment with an oversleeve partially extended;

FIG. 2d is a perspective view of the device embodiment with the oversleeve more fully extended; and

FIG. 2e is a perspective view of the device embodiment with the oversleeve yet more fully extended and a severed suture.

FIGS. 3a-3e are views of an alternative embodiment of a suture cutting device which includes an elongate member and a slidable member;

FIG. 3a is a side view of an embodiment of the device;

FIG. 3b is a top view thereof;

FIG. 3c is a cross-sectional view of the elongate member along line 3c-3c of FIG. 3a;

FIG. 3d is a perspective view of the slidable member; and

FIG. 3e is a perspective view of a cut-open cross section of the elongate member of the device.

### DETAILED DESCRIPTION OF THE DRAWINGS AND THE PRESENTLY PREFERRED EMBODIMENTS

Before providing a detailed description of embodiments of the invention, it may be useful to provide some definitions of terms as they are used in this specification and the claims thereof.

As used below, the term “endoscopic” and its grammatical variants are defined in a broader than usual sense to include endoscopes, laparoscopes, arthroscopes, and other minimally invasive surgical devices currently in use or to be developed in the future. Specifically, traditional medical usage of the term “endoscope” refers to a surgical or diagnostic instrument used through an existing body orifice while laparoscopes, arthroscopes, and the like are used through body apertures created by one or more incisions. There are other distinctions in techniques using these instruments that do not significantly affect use of the suture cutting device embodiments described and claimed herein. To avoid redundancy of the phrase “endoscopes, laparoscopes, arthroscopes, anuscopes, and the like” and variants thereof, this specification uses “endoscope” and variants of that term (for example, “endoscopic,” “endoscopy”) in a generic and inclusive manner. For the purposes of this application, “endoscope” and terms derived therefrom are defined to include not only a traditional endoscope, but also to include any such currently existing or future-invented minimally invasive surgical devices (for example, to include laparoscope, arthroscope, pelviscope, duodenoscope, hysteroscope, etc.) that utilize a natural or incised opening in a body to introduce tools into a body lumen through an aperture.

In this specification, the term “manipulating” and variants thereof are defined to include movement or cutting.

The term “body lumen” means any open area inside a body, whether existing naturally (for example, a vessel such as a blood vessel or bodily passage such as a portion of the alimentary canal) or being created by manipulation (for example, an open space within or beside an organ created by movement of a surgical tool).

The terms “has,” “having,” and “including” and their respective grammatical variants as used in the description and claims are defined to be open and inclusive, equivalent in meaning to the term “comprising” as that term is regularly interpreted in patent law and practice.

In one aspect, the claimed embodiments include a device for manipulating a suture in a body lumen by means of an elongate member. In one embodiment, the elongate member is appropriately sized for introduction into the body lumen in conjunction with an endoscope and has a proximal end and a distal end. In another embodiment, the elongate member is configured for general (including non-endoscopic) surgical use. In addition, the elongate member has at least one indented surface wherein is disposed a cutting edge. Some embodiments of the invention are configured for single use and disposal; other embodiments are suitable for sterilization and re-use.

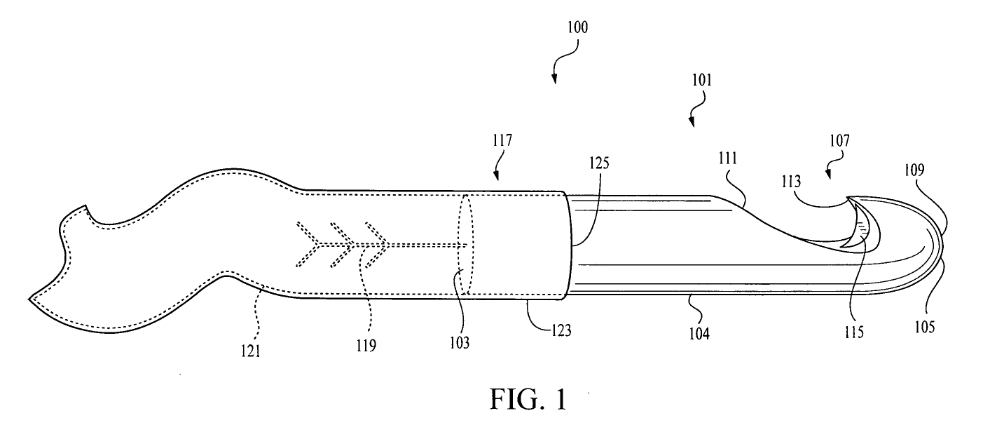

FIG. 1 shows an embodiment of a device 100 for manipulating a suture in a body lumen including an elongate member illustrated in this embodiment as a substantially cylindrical shaft 101. In this embodiment, the diameter of shaft 101 is from about 2 to about 3.5 millimeters and the length of the catheter is about 5 centimeters; however, other embodiments are likely to be a different size depending upon the application and uses for which those embodiments are intended. The shaft 101 has a proximal end 103 connected by a main body 104 to a distal end 105. The shaft 101 also has an indented surface. In the embodiment illustrated in FIG. 1, the indented surface forms a hook 107 near the distal end 105. The hook 107 is defined by an outer arched surface 109 and an inner arched surface 111. The two surfaces 107, 109 meet at an extremity 113 that projects substantially toward the proximal end 103.

In this embodiment, the inner arched surface 111 has disposed within it a cutting edge, illustrated in this embodiment as a surgical-grade steel blade 115. The sharp surface of the blade 115 is oriented toward the proximal end 103 and does not project beyond the outer periphery of the shaft 101. In various other embodiments, the blade 115 may be disposed by adhesive or other connecting means to the surface of the inner arched surface 111, or it may be partially embedded in the inner arched surface 111 by affixation into a cavity, or it may be affixed in some other way (for example, using a mechanical structure such as a screw, pin, rivet, or the like, or welding, adhesive, or some other appropriate mechanism). Alternatively, the blade 115 may be disposed in the inner arched surface 111 as an integral part of the elongate member. This could be accomplished, for example, by using an overmolding process wherein the material used to compose an elongate member, such as the illustrated embodiment of the shaft 101, is molded around the blade 115.

The more proximal portion of the shaft 101 not comprising the hook 107 forms a shank 117. In this embodiment, the proximal end 103 includes an attachment structure 119 anchoring the shaft 101 to a flexible member 121. As illustrated in this embodiment, the attachment structure 119 is a barbed member capable of securely anchoring shaft 101 to the flexible member 121. The flexible member 121 of this embodiment provides a flexible structure for inserting the catheter into a body lumen and for manipulating the shaft 101 through an endoscope. In the illustrated embodiment, the flexible member 121 is a flexible rod capable of translating movement at its proximal end into longitudinal or rotational movement of the shaft 101 attached at its distal end. In alternative embodiments, the flexible member 121 may be, for example, a catheter shaft, wire, rod, or another manipulable elongate structure.

In the illustrated embodiment of FIG. 1, the device also includes a component for opposing the cutting edge, including a leading surface that is resistant to deforming force, which is illustrated in this embodiment as an oversleeve 123, where the oversleeve 123 is frictionally slidable over the shaft 101. For the purposes of the illustrated embodiment, the oversleeve 123 includes at least one surface, preferably a leading surface 125, in slidable frictional contact with the external surface of the shaft 101. In the illustrated embodiment, the frictional contact is such that the distance between the leading surface 125 of the oversleeve 123 and the external surface of the shaft 101 is less than the diameter of a surgical suture to be severed.

The shaft 101 of the embodiment illustrated in FIG. 1, along with other potential embodiments of the overall device, may be made by a variety of methods including but not limited to using an injection molding process, an overmolding process, a casting process, a machining process, a combination thereof, or any later developed technologies/methods. Suitable materials for composing the elongate member include but are not limited to plastics and metallic alloys. In one embodiment, an elongate member as illustrated by the shaft 101 is formed by an overmolding process wherein the blade 115 is placed in a mold and a surgical grade plastic is cast around it to form a full shape of the shaft 101 such that the blade 115 is disposed in a molded indentation (e.g., inner arched surface 111). In other embodiments, the indented surface may be formed by a machining process or other process that removes material from the body of the elongate member to form an indentation. Alternatively, the indented surface may be formed by use of a molding process during formation of the elongate member, by deforming a surface of the elongate member to form an indentation, or by any other means suitable for forming an indentation in the construction material of the elongate member. The cutting structure may be disposed in or mounted to the indented surface before, during, or after the formation of the indented surface, as is appropriate to the method of formation and the desired means of disposing the cutting structure to the indented surface. Other embodiments of the suture cutting device may also be made by the above methods.

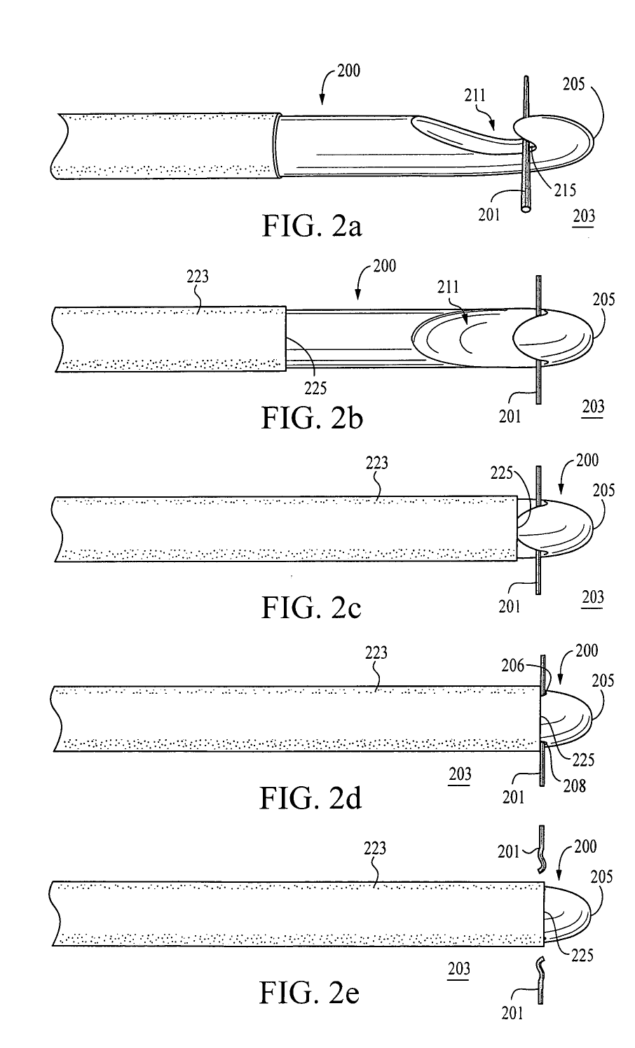

FIGS. 2a-2e show an embodiment of a method for cutting a suture. As illustrated, this embodiment of the method includes the steps of: (1) endoscopically positioning an elongate member comprising an indented surface, and further comprising a cutting edge, such that a suture transverses a path of the cutting edge; (2) moving a component for opposing the cutting structure, which is in frictional contact with the elongate structure such that a leading surface of the component for opposing the cutting structure frictionally contacts the suture against at least two points of the elongate structure wherein at least one of the at least two points is not on the same side of the cutting structure as another of the at least two points; (3) further moving the component for opposing the cutting structure such that the frictional contact at the at least two points draws the suture taut across the cutting edge; and (4) moving yet farther the component for opposing the cutting structure such that the tautness of the suture across the cutting structure severs the suture.

FIGS. 2a-2e specifically show a embodiment for a method of endoscopically cutting a suture. FIG. 2a is a side view of an embodiment of a suture cutting device, positioned appropriately for manipulating a suture 201. In an embodiment of a method for endoscopically cutting a suture, the illustrated device has been endoscopically introduced into a body lumen, such as a portion of the gastrointestinal tract, and inserted between a suture 201 and underlying tissue 203 by directing a distal end 205 of the device body 200 therebetween. The device body 200 is positioned such that suture 201 is lying transversely across an indented surface 211. In the illustrated embodiment, the suture 201 is positioned adjacent a blade 215. FIG. 2b is a top view of the embodiment illustrated in 2a. FIG. 2c illustrates that, in this embodiment, a leading surface 225 of an oversleeve 223 is extended toward the distal end 205 so as to enclose the indented surface 211 partially within the oversleeve 223. FIG. 2d shows the oversleeve 223 extended more distally than in FIG. 2c. As shown in FIG. 2d, because the leading surface 225 is in slidable frictional contact with the device body 200, the leading surface 225 pinches the suture 201 against the indented surface 211 at two contact points 206, 208. The frictional contact at the contact points 206, 208 draws the suture 201 taut across cutting edge of the blade 215. As illustrated in FIG. 2e, when the leading surface 225 of the oversleeve 223 is extended more distally, an increased tension of the suture 201 across the blade 215 severs the suture 201. The amount of control and the gentleness possible with the described motion in this embodiment significantly reduces a potential risk of the sutures 201 tearing or otherwise harming the underlying tissue 203. Alternatively, the suture 201 may be cut more directly by exerting proximally directed force such that the blade 215 is pulled against the suture 201 to sever it without the presence of the oversleeve 223.

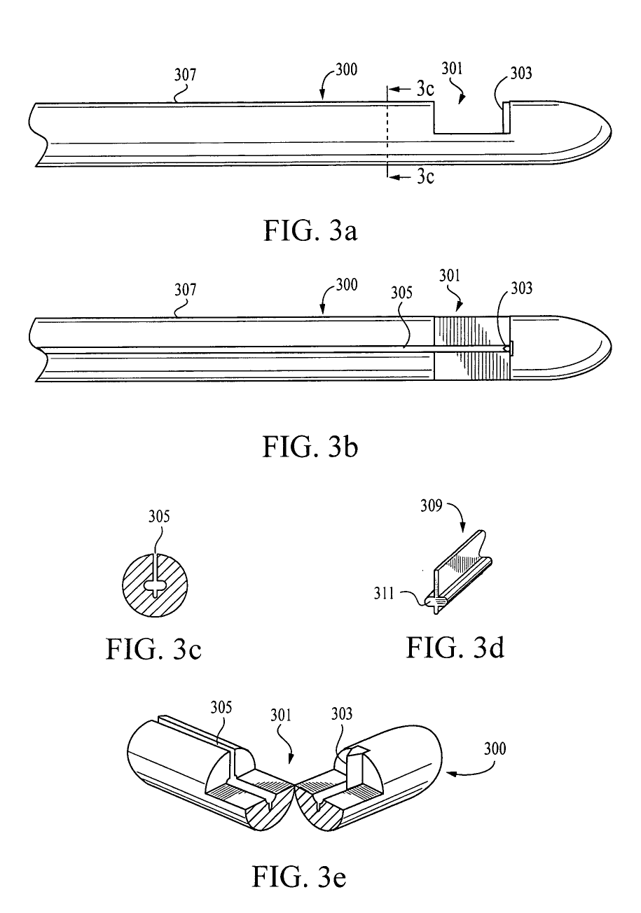

The embodiment of a suture cutting device illustrated in FIGS. 3a-3d features an indented surface that is angular rather than arched or curvilinear. In this illustrated embodiment, the component for opposing the cutting structure is a slidable member that is guided down a channel disposed in an anterior surface of the elongate member. In an alternative embodiment, the channel is an enclosed lumen of the elongate member. The slidable member may be advanced such that it will severingly pinch a suture against the cutting structure disposed in the indentation of the elongate member, thereby cutting the suture. Embodiments including a slidable member as described and illustrated may have an indentation that is angular, arched, curvilinear or another shape amenable to holding a blade in position for cutting a suture.

FIG. 3a is a side view of the angular-indentation suture cutting device. The device body is an elongate member 300, which has an angular indentation 301 wherein is disposed a cutting structure 303. As illustrated, a cutting structure 303 is mounted in the center of the distal end of the angular indentation 301. FIG. 3b is a top view of the device, illustrating the relative position of a central channel 305 along a dorsal surface 307. The dorsal surface 307 is on the same side of the elongate member 300 as is the indentation 301. FIG. 3c is a transverse cross-sectional view of the elongate member 300 along line 3c-3c. The central channel 305 serves as a passage for a slidable member 309. As illustrated, the channel 305 is open to the dorsal surface 307. In a different embodiment, channel 305 may be enclosed, and be formed as a lumen through elongate member 300. In another alternate embodiment, channel 305 may be off-center.

In the illustrated embodiment, the slidable member 309 may be advanced through the channel 305 to severingly pinch a suture against the cutting structure 303. FIG. 3d is a perspective view of the slidable member 309. The slidable member 309 has a smooth, flat end surface 311. Alternative embodiments of the slidable member 309 may have, for example, textured, grooved, curvilinear, or angled surfaces. In the illustrated embodiment, the end surface 311 acts as a leading surface and may be advanced through the channel 305 toward the distal end 313 of the elongate member 300. In one application of the illustrated embodiment, the elongate member 300 may be positioned such that a suture lies across the indentation 301. The slidable member 309 may then advanced through the channel 305. The surface 311 of the slidable member 309 may then force the suture against the cutting structure 303, severing the suture. In an embodiment where the end surface 311 is flat and smooth, the suture is pushed directly against the cutting structure 303.

FIG. 3e is a perspective view of a cut-open cross section of the elongate member 300. The elongate member 300 has an angular indentation 301 wherein is disposed a cutting structure 303. As illustrated, the cutting structure 303 is mounted in the center of distal end of indentation 301. As illustrated, channel 305 is open to the dorsal surface. In an alternate embodiment, either the channel 305, or the cutting structure 303, or both are off-center. In one embodiment, the surface 311 is composed of the same material as the body of the slidable member 309. In another embodiment, the surface 311 is composed of a different material than the body of slidable member 309. In still another embodiment (not shown), the cutting structure 303 is disposed on the end surface 311 rather than in the indentation 301 of the elongate member 300.

In one embodiment, the claimed device is configured for use in a general surgical setting for cutting a suture. In this embodiment, the proximal end of the elongate member containing a blade in an indentation may be affixed to, or integral with a flexible, inflexible, or semi-flexible structure for manipulating the distal end. Alternatively, the proximal end of the elongate member may itself sufficiently long to be useful by direct manipulation. In this embodiment, a component for opposing the cutting structure such as an oversleeve, a wire, a slidable member, or an equivalent thereof, having at least a leading surface is also used. To cut a suture, the distal end of the elongate member is guided under a suture such that the suture lies across the indentation containing the cutting edge. Then, the leading surface of the component for opposing the cutting structure may be guided along the elongate member in a manner that forces the suture across the cutting edge, cutting the suture.

It is therefore intended that the foregoing detailed description be regarded as illustrative rather than limiting, and that it be understood that following claims, including all equivalents, are intended to define the spirit and scope of this invention.

1. A minimally invasive medical device for cutting a suture, comprising:
an elongate member, comprising:
a proximal end, a distal end, a main body extending between the proximal and distal ends, and an indented surface near the distal end; and
a cutting structure, including a cutting edge, disposed in the indented surface.

2. The device of claim 1, further comprising a first structure for attaching the proximal end to a second structure configured for inserting the elongate member into a body lumen.

3. The device of claim 1, wherein the cutting structure is mounted such that at least one portion of the cutting structure protrudes beyond the indented surface but does not protrude beyond an outermost periphery of the elongate member.

4. The device of claim 1, further comprising:
a component for opposing the cutting edge, the component comprising a leading surface and being slidable along a surface of the elongate member, wherein the leading surface is resistant to deforming force; and
a mechanism configured for advancing the component for opposing the cutting structure toward the cutting edge.

5. The device of claim 1, wherein the indented surface comprises a curvilinear surface.

6. The device of claim 1, wherein the indented surface comprises an angular surface.

7. The device of claim 1, wherein the cutting structure is a surgical grade steel blade.

8. A method for making the device of claim 1, comprising:
forming an elongate member which comprises:
a proximal end, a distal end, and a main body extending between the proximal and distal ends;
forming an indented surface in the elongate member; and
disposing a cutting structure, including a cutting edge, in the indented surface.

9. The method of claim 8 wherein the cutting structure is a surgical grade steel blade.

10. The method of claim 8 wherein forming the elongate member comprises machining the elongate member from a metal.

11. The method of claim 8 wherein forming the elongate member comprises molding the elongate member from surgical grade plastic.

12. The method of claim 8 wherein forming the indented surface comprises shaping an indented surface in the elongate member.

13. The method of claim 8 wherein forming the elongate member comprises molding the elongate member in a cast shaped to form an indented surface.

14. The method of claim 8 wherein disposing a cutting structure in the indented surface comprises forming the elongate member around the cutting structure such that the cutting structure is disposed in the indented surface.

15. The method of claim 8 wherein disposing a cutting structure in the indented surface comprises forming the cutting structure from a material of the elongate member such that the cutting structure is disposed in the indented surface.

16. The method of claim 8 wherein disposing a cutting structure in the indented surface comprises affixing the cutting structure in the indented surface.

17. A method for cutting a suture in a body lumen, comprising:
a) positioning a device according to claim 4 near a suture;
b) positioning the distal end of the elongate member such that the suture traverses the indented surface adjacent the cutting edge;
c) advancing the component for opposing the cutting structure toward the cutting structure such that the leading surface directs the suture into contact against the cutting edge; and
d) further advancing the component for opposing the cutting structure such that force from the leading-surface-directed contact of the suture against the cutting structure severs the suture.

18. A method for cutting a suture in a body lumen, comprising:
a) introducing a device according to claim 4, in conjunction with a minimally invasive surgical device, into a body lumen;
b) positioning the device near a suture;
c) positioning the distal end of the elongate member such that the suture traverses the indented surface;
d) advancing the component for opposing the cutting structure toward the distal end such that the leading surface directs the suture into contact against the cutting edge; and
e) further advancing the component for opposing the cutting structure such that the leading surface directed contact of the suture against the cutting structure severs the suture.

19. The method of claim 18 wherein the minimally invasive surgical device is selected from a group consisting of: anuscope, arthroscope, bronchoscope, choledoscope, colonoscope, cystoscope, duodenoscope, earscope, endoscope, endotrachealscope, esophagoscope, hysteroscope, laparoscope, laryngoscope, nasosinuscope, nephroscope, otoscope, pancreatoscope, pelviscope, proctorscope, rectoscope, resectoscope, rhinoscope, sigmoidoscope, sinuscope, thoracoscope, and ureteroscope.

20. A method for cutting a suture, comprising:
a) positioning the device of claim 4 near a suture;
b) positioning the distal end of the elongate member such that the suture traverses a path of the cutting structure in the indented surface;
c) advancing the component for opposing the cutting structure such that the leading surface of the component for opposing the cutting structure frictionally contacts the suture against at least two points of the elongate member wherein at least one of the at least two points is not on the same side of the cutting structure as another of the at least two points;
d) further advancing the component for opposing the cutting structure such that the frictional contact at the at least two points draws the suture taut across the cutting edge; and
yet further advancing the component for opposing the cutting structure such that the tautness of the suture across the cutting structure severs the suture.

21. A device for manipulating a suture, comprising:
a substantially cylindrical catheter, comprising:
a proximal end, a rounded distal end, and a main body extending between the proximal and distal ends;
an indented surface near the distal end, having an arched shape wherein is disposed a cutting structure having a cutting edge,
the cutting structure being affixed in a distal portion of the arched shape such that the cutting edge does not protrude beyond an outermost periphery of the elongate member; and
the cutting edge being oriented toward the proximal end;
being sized and shaped for introduction into a body lumen in conjunction with a minimally invasive surgical device;
a structure configured for inserting the catheter into a body lumen attached to the proximal end of the catheter; and
an oversleeve comprising a leading surface shaped and sized so as to be frictionally slidable over the outer surface of the catheter.
